(19) United States
(12) Patent Application Publication
(10) Pub. No.: US 2025/0053791 A1
(43) Pub. Date: Feb. 13, 2025

(54) METHOD TO EVALUATE AND FACT-CHECK AN AI LARGE LANGUAGE MODEL CHAT RESPONSE USING DOMAIN- SPECIFIC AND DOMAIN-AGNOSTIC GUIDANCE FOR PERSONALIZED MEDICAL PROVIDER-PATIENT CONSULT

(71) Applicant: NORTHWESTERN UNIVERSITY, Evanston, IL (US)

(72) Inventors: Peng Thian Troy Teo, Evanston, IL (US); Amulya Yalamanchili, Evanston, IL (US); Bishwambhar Sengupta, Evanston, IL (US); Tarita Thomas, Evanston, IL (US); Bharat B. Mittal, Evanston, IL (US); Mohamed Abazeed, Evanston, IL (US)

(21) Appl. No.: 18/773,685

(22) Filed: Jul. 16, 2024

Related U.S. Application Data
(60) Provisional application No. 63/531,036, filed on Aug. 7, 2023.

(51) Int. Cl. G06N 3/0455; G16H 80/00

(52) U.S. Cl. CPC G06N 3/0455; G16H 80/00

(57) ABSTRACT

A method for evaluating an artificial intelligence (AI) large language model (LLM) generated response, the method comprising receiving a user query in the form of patient-related questions for a medical treatment domain; analyzing the user query using a LLM learned with open-source data and outputting a LLM answer from the LLM; performing a domain-specific evaluation of the LLM answer; performing a domain-agnostic evaluation of the LLM answer; generating at least one metric for the LLM answer based on the domain-specific evaluation and the domain-agnostic evaluation of the LLM answer; and evaluating the quality of the LLM answer based on the at least one metric.

### CROSS-REFERENCE TO RELATED PATENT APPLICATION

This application claims priority to and the benefit of U.S. Provisional Patent Application Ser. No. 63/531,036, filed Aug. 7, 2023, which is incorporated herein in its entirety by reference.

### FIELD OF THE INVENTION

The present disclosure relates generally to the field of information analysis. More specifically, the present invention relates to the field of verifying the quality of statements and responses generated by an artificial intelligence (AI) large language model (LLM) chatbot.

### BACKGROUND OF THE INVENTION

The background description provided herein is for the purpose of generally presenting the context of the present invention. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions.

ChatGPT is a large language model (LLM) trained on very large text datasets in order to interpret text input and respond in a conversational manner. It has shown promise in answering test questions for medical school and specialty boards, simplifying radiology reports, and writing research abstracts. AI models like ChatGPT have the potential to alter medical practice and improve efficiency by reducing workload and optimizing performance. By leveraging the ability of AI, health care providers may be able to reallocate resources and attention to more complex tasks. However, notable concerns surrounding ChatGPT are the reliability of its training data and processes, which can in some cases lead to the generation of factually inaccurate responses or ‘hallucinations’. In medical applications, where patient safety is the priority, the ability to evaluate the quality of responses generated by ChatGPT is pivotal and remains the rate-limiting threshold for implementation.

Radiation oncology is a highly complex patient facing specialty that utilizes specialized machinery and technology and expertise from multiple disciplines including clinical medicine, physics, and dosimetry. The clinical practice workflow in radiation oncology incorporates several tasks, some which involve the patient directly. The patient facing steps in typical workflow include a consultation with a radiation oncologist, simulation for treatment planning, treatments facilitated by radiation therapists, and on-treatment and follow-up visits with a radiation oncologist. There are also multiple steps not directly involving patient participation, including treatment planning to optimize radiation delivery. In addition, radiation oncology uses specialized terminology, equipment, and technology that are often not familiar to patients and other medical providers in other fields. About one-third of the words used during an initial consultation is medical jargon, and another one-third are common words that may have different meanings when pertaining to radiation treatments. The complex processes involved can be intimidating or overwhelming to patients, and can lead to patient anxiety, poor understanding, and difficulty making treatment decisions and adhering to treatments. During the initial consultation as well as throughout treatment, the clinician must determine how to best disseminate information in a comprehensible but complete manner. Patients often have many follow up questions involving the treatment process, side effects and safety, and how treatments are designed and delivered. These factors collectively contribute to the complexities and challenges associated with achieving effective patient-physician communication.

Oncologic treatments cause several physical and psychological effects, which patients may find difficult to discuss with their provider. Sensitive questions may be particularly relevant in female- and male-associated cancers in the breast, gynecologic sites, and the prostate. Treatments including surgery, systemic therapy, and radiotherapy each cause physical changes that can impact body image as well as side effects that alter sexual health and function. The most common patient barrier to discussion of sensitive topics like sexual function is discomfort and reluctance in initiating discussions, and many patients do not receive sufficient sexual health care. Patient communication with a non-sentient chatbot can clearly lower these barriers.

Provider-patient communication is vitally important in radiation oncology, and patients often have many follow up questions following the initial consultation and throughout treatment. As providers are increasingly expected to be virtual, patient messages consume a high proportion of clinical staff work hours. The availability of the radiation oncology team for patient questions and discussions is often limited by other demands on provider time, including documentation requirements related to the electronic health record and insurance prior authorizations. These burdens have been linked to physician burnout and reduction in quality of clinical care.

To improve patient education and better prepare patients for treatment, interventions such as providing an educational video prior to initial physician consultation, or including an additional patient-physicist consultation, have been proposed. Additionally, there are several online resources from reputable medical organizations that have sought to answer common patient questions. Due to the variety of cancer subsites and treatment modalities, these question-and-answer resources often do not provide tailored materials applicable to a particular patient. There is also high variability between websites in depth and breadth of information provided. Online education materials from academic radiation oncology websites as well as from major radiation professional websites have been found to be more complex than is recommended by the National Institute of Health, US Department of Health and Human Services, and American Medical Association.

As electronic communication is increasingly utilized by patients, chatbots such as ChatGPT can help play a future role in patient-provider communication by empowering patients and alleviating some burden on providers. However, ChatGPT was not specifically trained to answer oncology related patient questions, raising the question of whether ChatGPT generated responses are accurate, complete, and without potential harm to patients. No current literature has examined the ability of ChatGPT to answer common patient questions related to radiation treatments.

Therefore, there remains an imperative need for utilizing a LLM to provide answers to questions that are commonly encountered in consultations and communication between patient and radiation oncology physician, and a method for evaluating the quality, confidence, and efficacy of the answers provided by the LLM in the field of radiation oncology.

### SUMMARY OF THE INVENTION

In light of the foregoing, this invention evaluates the potential of utilizing ChatGPT to provide answers to questions that are commonly encountered in an initial consultation between patient and radiation oncology physician. Since many common patient questions are found on websites and training materials provided by professional society websites, the relative performance of ChatGPT and its responses will be compared to information provided by these sites. A quantitative evaluation methodology involving both domain-specific expertise/knowledge and domain-agnostic metrices are introduced. While domain-specific metrics rely on expert guidance and human-in-the-loop evaluation to assess the quality, accuracy, and potential harm of ChatGPT's responses, computationally generated domain-agnostic metrics are automatically computed based on statistical analysis of the text in ChatGPT's responses. This combination of expert-driven and data-driven evaluation approaches provides a comprehensive assessment of ChatGPT's performance.

In one aspect of the invention, a method for evaluating an artificial intelligence (AI) large language model (LLM) generated response comprises generating a LLM answer for a user query by a LLM; performing a domain-specific evaluation of the LLM answer; and performing a domain-agnostic evaluation of the LLM answer, wherein the user query is in a form of patient-related question in a medical treatment domain.

In one embodiment, the process of generating the LLM answer comprises receiving the user query; and analyzing the user query using the LLM, wherein the LLM is learned with open-source data.

In one embodiment, the processes of performing the domain-specific evaluation of the LLM answer and the domain-agnostic evaluation of the LLM answer are automated.

In one embodiment, the method further comprises generating at least one metric for the LLM answer from the domain-specific evaluation and the domain-agnostic evaluation of the LLM answer; and evaluating the quality of the LLM answer based on the at least one metric.

In one embodiment, the method further comprises initiating a human-in-the-loop process to evaluate the at least one metric generated from the domain-specific evaluation.

In one embodiment, the medical treatment domain comprises medical oncology or radiation oncology.

In one embodiment, the domain-specific evaluation performs a fact-checking of the LLM answer.

In one embodiment, the domain-agnostic evaluation checks relevance of the LLM answer to general public's understanding of the LLM answer.

In one embodiment, the domain-specific evaluation comprises comparing the LLM answer with a reference answer identified by a group of human domain-specific experts.

In one embodiment, a similarity metric is computed to compare the similarity between the LLM answer and the reference answer.

In one embodiment, a set of domain-specific evaluation metrics is used in the comparison of the LLM answer and the reference answer.

In one embodiment, the set of domain-specific evaluation metrics comprises potential harm, factual correctness, completeness, and conciseness.

In one embodiment, each of the domain-specific evaluation metrics is graded with a numeric reference between 1 to 5 in the comparison of the LLM answer and the reference answer.

In one embodiment, the grade of each of the domain-specific evaluation metrics and the similarity metric between the LLM answer and reference answer are stored in a lookup table.

In one embodiment, the domain-agnostic evaluation comprises determining at least one readability metric; wherein the readability metric comprises an estimated school grade level required to understand the LLM answer.

In one embodiment, the domain-agnostic evaluation comprises determining a sentence complexity for the LLM answer.

In one embodiment, the domain-agnostic evaluation comprises determining a readability score for the LLM answer.

In one embodiment, the readability score detects a literacy bias of the LLM answer against a patient whose literacy is below an average literacy of the general public.

In one embodiment, the method further comprises mitigating the literacy bias against the patient's literacy by inputting into the LLM the readability score associated with the LLM answer; and requesting the LLM to generate a modified LLM answer with a modified readability score lower than the readability score.

In one embodiment, the domain-agnostic evaluation comprises determining a syllable score; wherein the syllable score comprises a syllable count in the LLM answer.

In one embodiment, the syllable score detects a syllable bias of the LLM answer against a patient with reading disorders.

In one embodiment, the method further comprises mitigating the syllable bias of the LLM answer against the patient with reading disorders by inputting the syllable score for the LLM answer into the LLM and requesting the LLM to generate a modified LLM answer with a lower syllable count.

In one embodiment, the domain-agnostic evaluation comprises determining a score for word count or number of sentences; wherein the score for word count or number of sentences determines an ease level of comprehension and interpretation of the LLM answer.

In one embodiment, the domain-agnostic evaluation comprises determining a lexicon score; wherein the lexicon score determines a subjectivity and contextual impact of the LLM answer.

In one embodiment, the method further comprises converting the user query and the LLM answer into a vectorized user query and a vectorized LLM answer, respectively; comparing the vectorized user query with a database of reference queries comprising more than one reference queries; computing a vectorized query similarity metric between the vectorized user query and each of the reference queries; selecting a surrogate user query from the reference queries wherein the surrogate user query has highest vectorized query similarity metric; returning the reference answer that corresponds to the surrogate user query, wherein the reference answer is a surrogate reference answer to the vectorized user query; and computing a vectorized answer similarity metric between the vectorized LLM answer with the surrogate reference answer.

In yet another aspect of the invention, a non-transitory computer readable medium storing a program causing a computer to execute a process for evaluating an artificial intelligence (AI) large language model (LLM) generated response comprises generating a LLM answer for a user query by a LLM; performing a domain-specific evaluation of the LLM answer; and performing a domain-agnostic evaluation of the LLM answer, wherein the user query is in a form of patient-related question in a medical treatment domain.

In one embodiment, the process of generating the LLM answer comprises receiving the user query; and analyzing the user query using the LLM, wherein the LLM is learned with open-source data.

In one embodiment, the processes of performing the domain-specific evaluation of the LLM answer and the domain-agnostic evaluation of the LLM answer are automated.

In one embodiment, the non-transitory computer readable medium further comprises generating at least one metric for the LLM answer from the domain-specific evaluation and the domain-agnostic evaluation of the LLM answer; and evaluating the quality of the LLM answer based on the at least one metric.

In one embodiment, the non-transitory computer readable medium further comprises initiating a human-in-the-loop process to evaluate the at least one metric generated from the domain-specific evaluation.

In another aspect of the invention, a method comprising receiving a user query in the form of patient-related questions for a particular medical treatment domain such as medical oncology or radiation oncology; analyzing the user query using a large machine learning large language model (LLM) learned with open-source data and outputting an answer from the LLM; performing a domain-specific evaluation of the LLM answer wherein the domain-specific evaluation is for the purpose of fact-checking the LLM answer; performing a domain-agnostic evaluation of the LLM answer wherein the domain-agnostic evaluation is for the purpose of checking the relevance of the LLM answer to the understanding of the general public, i.e. not a domain expert; automating the process of performing a domain-specific and a domain-agnostic evaluation of the LLM answers triggering a human-in-the-loop process to evaluate the domain-specific evaluation metric should the automated approach of evaluating the domain-specific evaluation metric fails; generating a metrics and/or metrics for the LLM answer wherein the purpose of the metrics is to provide an indication of confidence and quality of the LLM answer

In one embodiment, the domain-specific evaluation approach involves comparing the LLM answer with a reference answer identified by a group of human domain-specific experts.

In one embodiment, a similarity metric is computed to compare the similarity between the LLM and the reference answer.

In one embodiment, a set of domain-specific evaluation metrics identified by a group of human domain-specific experts is used to compare the LLM and the reference answer. As an example, for the use case of fact-checking medical/health-related answers, the domain-specific evaluation metrics include potential harm, factual correctness, completeness, and conciseness.

In one embodiment, rankings are provided for each of the evaluation metrics when comparing LLM answers with respect to the reference answers. As an example, a Likert scale with 5=Much better, 4=Somewhat better, 3=the same, 2=Somewhat worse, 1=Much worse is used to determine the potential harm, factual correctness, completeness, and conciseness of the LLM answers when compared to the reference answer. The rankings are provided by a group of domain-specific experts.

In one embodiment, the ranking for each of the evaluation metrics, along with the similarity metric between the corresponding LLM and reference answers are stored in a lookup table, wherein the lookup table facilitates the estimation of the ranking of the evaluation metrics by using the similarity metric computed between subsequent new LLM answers and their corresponding reference answers.

In one embodiment, the domain-agnostic evaluation approach involves computing various readability metrics domain-specific evaluation approach. The readability metrics return the estimated school grade level required to understand the text. The readability metrics can be used to determine whether the LLM answers met professional guidelines suggesting that, for example, medical/health information be presented at a sixth-grade reading level. The readability metric can also be used to detect whether LLM answers are biased against users/patients with low education and low-income levels, and low mental cognition capability.

In one embodiment, the domain-agnostic evaluation approach involves determining the sentence complexity of the LLM answers by computing, as an example, the number of syllables, lexicon, sentences, and words in the LLM answers. The readability metric can be used to determine whether LLM answers are biased against users/patients with reading disorders, low mental cognition capability, and old age.

In one embodiment, the domain-agnostic evaluation approach involves determining a score, for example, a readability score for the LLM answers that determine the literacy and readability of English text for a foreign learner of English, or someone with a low level of education.

In one embodiment, the domain-agnostic approach is used to detect and resolve the biases of LLM answers against users/patients whose first language is not English.

In one embodiment, a method to mitigate the biases against literacy would involve inputting into the LLM the readability score associated with the current LLM answers and requesting LLM to generate a new answer with either a readability score lower than the current one, or for a lower literacy level reader.

In one embodiment, the domain-agnostic evaluation approach involves determining a score, for example, a syllable score that counts the number of syllables in the LLM answers.

In one embodiment, the syllable score could be used to detect and resolve the biases and inefficacy of LLM answers against users/patients with deficits in phonological and phonemic awareness, and difficulty in reading words with more than one syllable, polysyllabic words whose symptoms are often associated with reading disorders such as dyslexia.

In one embodiment, a method to mitigate the biases of LLM answers against readers with reading disorders would involve inputting the syllable score for the current LLM answer into the LLM and requesting the LLM to generate a new answer with a lower syllable count.

In one embodiment, the domain-agnostic evaluation approach involves determining a score, for example, a word count or number of sentences. The score would help to determine the ease of comprehension and interpretation of the generated LLM chatbot answers. Long sentences could be modified to help improve the reading comprehension of patients.

In one embodiment, the domain-agnostic evaluation approach involves determining a score, for example, a lexicon score. The score would help to determine the vocabulary of a language or branch of knowledge, such as medical knowledge. Knowing the scores would allow the determination of the subjectivity and contextual impact of the chatbot answers, which in turn, would determine the ease of interpretation of the chatbot generated answers.

In one embodiment, the method further comprising converting both user query and LLM answers into vectorized tokens; comparing the vectorized user query with a database of known queries wherein the known queries are the reference queries; computing the similarity metric between the user query and each of the reference queries; selecting the reference query that has the highest similarity metric to be a surrogate of the user query, and returning the reference answer that corresponds to the selected reference query, wherein the reference answer is the surrogate reference answer to the user query; computing the similarity metric between the vectorized LLM answer with the surrogate reference answer.

In one embodiment, the similarity metric between the vectorized LLM answer with the surrogate reference answer is used as a scaling factor, i.e., multiplying the scaling factor to the pre-determined rankings of the domain-specific evaluation metric for the LLM answers specified in the lookup table.

In one embodiment, to determine the ranking of the domain-specific evaluation metric for the LLM answers, the similarity metric is compared to a threshold wherein: if similarity metric>threshold, obtain a new domain-specific rating for the LLM answer automatically by multiplying the similarity metric with the pre-determined domain-specific rating (specified in the lookup table) for the reference answer; and if similarity metric<threshold, trigger a group of human-in the-loop domain experts to manually determine a new set of domain-specific ratings for the LLM answer. Append this new domain-specific rating for the new LLM query into the existing lookup table of domain-specific ratings.

In one embodiment, either using the domain-specific, or domain-agnostic metric alone or an aggregated metric encompassing both metrics, an overall score will be generated to indicate the confidence level of the quality of the generated LLM answer.

In one embodiment, the use of the domain-specific and/or domain-agnostic metric could serve as a method to autonomously monitor the quality of both the queries entered into, and the answers generated by a web-based LLM model whose purpose is to provide medical, healthcare and cancer care information on behalf of the organization hosting the web pages.

In one aspect of the invention, a system comprising a processor configured to receive a user query in the form of patient-related questions for a particular medical treatment domain such as medical oncology or radiation oncology; analyze the user query using a large machine learning large language model (LLM) learned with open-source data and outputting an answer from the LLM; perform a domain-specific evaluation of the LLM answer wherein the domain-specific evaluation is for the purpose of fact-checking the LLM answer; perform a domain-agnostic evaluation of the LLM answer wherein the domain-agnostic evaluation is for the purpose of checking the relevance of the LLM answer to the understanding of the general public, i.e. not a domain expert; automate the process of performing a domain-specific and a domain-agnostic evaluation of the LLM answers; trigger a human-in-the-loop process to evaluate the domain-specific evaluation metric should the automated approach of evaluating the domain-specific evaluation metric fails; generate a metrics and/or metrics for the LLM answer wherein the purpose of the metrics is to provide an indication of confidence and quality of the LLM answer.

In another aspect of the invention, a computer program product embodied in a non-transitory computer-readable medium and comprising computer instructions for receiving a user query in the form of patient-related questions for a particular medical treatment domain such as medical oncology or radiation oncology; analyzing the user query using a large machine learning large language model (LLM) learned with open-source data and outputting an answer from the LLM; performing a domain-specific evaluation of the LLM answer wherein the domain-specific evaluation is for the purpose of fact-checking the LLM answer; performing a domain-agnostic evaluation of the LLM answer wherein the domain-agnostic evaluation is for the purpose of checking the relevance of the LLM answer to the understanding of the general public, i.e. not a domain expert; automating the process of performing a domain-specific and a domain-agnostic evaluation of the LLM answers; triggering a human-in-the-loop process to evaluate the domain-specific evaluation metric should the automated approach of evaluating the domain-specific evaluation metric fails; generating a metrics and/or metrics for the LLM answer wherein the purpose of the metrics is to provide an indication of confidence and quality of the LLM answer.

### BRIEF DESCRIPTION OF THE DRAWINGS

The accompanying drawings illustrate one or more embodiments of the invention and together with the written description, serve to explain the principles of the invention. Wherever possible, the same reference numbers are used throughout the drawings to refer to the same or like elements of an embodiment.

FIG. 1 depicts a flow chart reflecting the method and system of fact-checking the answers provided by the LLM chatbot using domain-specific and domain-agnostic evaluations.

FIG. 2 depicts a flow chart for comparing LLM answer with an existing database of reference answers, and a similarity metric between the LLM and the reference answer was generated.

FIG. 3. depicts an example implementation of domain-specific evaluation of LLM answers.

FIG. 4. illustrates an example implementation of the ranking of LLM answers according to domain-specific metrics such as potential harm, relative factual correctness, relative completeness, and relative conciseness compared to the reference answers.

FIG. 5. depicts an example implementation of using readability scores as a set of domain-agnostic evaluation metrics to detect the biasness of LLM answers against users' education levels.

FIG. 6. depicts an example implementation of automatically quantifying the complexity and/or conciseness of LLM answers by computing the number of syllables, lexicon, sentences, and words in the LLM answers.

FIG. 7 depicts an example implementation of automatically selecting the appropriate reference answers corresponding to a query entered using the similarity metric computed between the query requested and a database of reference questions.

FIG. 8 depicts an example of the automatically generated metrics catered for domain-agnostic evaluation.

FIG. 9 shows a Likert scale plot for potential harm of all ChatGPT generated responses.

FIG. 10A shows a Likert scale plot for relative factual correctness, relative completeness, and relative conciseness of ChatGPT generated responses compared to online resource expert answers in categories of general radiation oncology issue.

FIG. 10B shows a Likert scale plot for relative factual correctness, relative completeness, and relative conciseness of ChatGPT generated responses compared to online resource expert answers in categories of treatment modality specific answers.

FIG. 10C shows a Likert scale plot for relative factual correctness, relative completeness, and relative conciseness of ChatGPT generated responses compared to online resource expert answers in categories of treatment site specific answers.

FIG. 11A shows Likert scale plots for relative factual correctness, relative completeness, and relative conciseness of ChatGPT generated responses compared to online resource expert answers in subcategories of Ext Beam, Linac, SRS SBRT, and Proton within treatment modality specific answers.

FIG. 11B shows Likert scale plots for relative factual correctness, relative completeness, and relative conciseness of ChatGPT generated responses compared to online resource expert answers in subcategories of MR Linac, Gamma Knife, IMRT, IGRT within treatment modality specific answers.

FIG. 12A shows Likert scale plots for relative factual correctness, relative completeness, and relative conciseness of ChatGPT generated responses compared to online resource expert answers in each subcategory within treatment subsite specific answers regarding cancers in brain, H&N, and Prostate.

FIG. 12B shows Likert scale plots for relative factual correctness, relative completeness, and relative conciseness of ChatGPT generated responses compared to online resource expert answers in each subcategory within treatment subsite specific answers regarding cancers in cervical, thyroid, and lymphoma.

FIG. 12C shows Likert scale plots for relative factual correctness, relative completeness, and relative conciseness of ChatGPT generated responses compared to online resource expert answers in each subcategory within treatment subsite specific answers regarding cancers in colorectal, lung, and breast.

FIG. 12D shows Likert scale plots for relative factual correctness, relative completeness, and relative conciseness of ChatGPT generated responses compared to online resource expert answers in each subcategory within treatment subsite specific answers regarding cancers in esophageal, pancreatic, and anal.

FIG. 13 shows computationally generated metrics for ChatGPT generated responses in categories of general radiation oncology issues.

FIG. 14 shows computationally generated metrics for ChatGPT generated responses in each subcategory within treatment modality specific answers.

FIG. 15 shows computationally generated metrics for ChatGPT generated responses in each subcategory within treatment subsite specific answers.

### DETAILED DESCRIPTION OF THE INVENTION

The invention will now be described more fully hereinafter with reference to the accompanying drawings, in which exemplary embodiments of the invention are shown. This invention may, however, be embodied in many different forms and should not be construed as limited to the embodiments set forth herein. Rather, these embodiments are provided so that this invention will be thorough and complete, and will fully convey the scope of the invention to those skilled in the art. Like reference numerals refer to like elements throughout.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term is the same, in the same context, whether it is highlighted. It will be appreciated that same thing can be the in more than one way. Consequently, alternative language and synonyms may be used for any one or more of the terms discussed herein, nor is any special significance to be placed upon whether a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the invention has been specifically disclosed by preferred embodiments and optional features, modification and variation of the concepts herein disclosed may be resorted to by those skilled in the art, and that such modifications and variations are considered to be within the scope of this invention as defined by the appended claims.

Whenever a range is given in the specification, for example, a temperature range, a time range, or a composition or concentration range, all intermediate ranges and subranges, as well as all individual values included in the ranges given are intended to be included in the invention. It will be understood that any subranges or individual values in a range or subrange that are included in the description herein can be excluded from the claims herein.

It will be understood that, as used in the description herein and throughout the claims that follow, the meaning of “a”, “an”, and “the” includes plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the invention.

Furthermore, relative terms, such as “lower” or “bottom” and “upper” or “top,” may be used herein to describe one element's relationship to another element as illustrated in the figures. It will be understood that relative terms are intended to encompass different orientations of the device in addition to the orientation depicted in the figures. For example, if the device in one of the figures is turned over, elements described as being on the “lower” side of other elements would then be oriented on “upper” sides of the other elements. The exemplary term “lower”, can therefore, encompasses both an orientation of “lower” and “upper,” depending of the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

It will be further understood that the terms “comprises” and/or “comprising”, or “includes” and/or “including”, or “has” and/or “having”, or “carry” and/or “carrying”, or “contain” and/or “containing”, or “involve” and/or “involving”, “characterized by”, and the like are to be open-ended, i.e., to mean including but not limited to. When used in this disclosure, they specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the invention, and will not be interpreted in an idealized or overly formal sense unless expressly so defined herein.

As used in the disclosure, “around”, “about”, “approximately” or “substantially” shall generally mean within 20 percent, preferably within 10 percent, and more preferably within 5 percent of a given value or range. Numerical quantities given herein are approximate, meaning that the term “around”, “about”, “approximately” or “substantially” can be inferred if not expressly stated.

As used in the disclosure, the phrase “at least one of A, B, and C” should be construed to mean a logical (A or B or C), using a non-exclusive logical OR. As used herein, the term “and/or” includes any and all combinations of one or more of the associated listed items.

### Methods

### Question-Answer Database

In one embodiment, question-answer resources from the websites of four large oncology and radiation oncology groups were assessed for the present invention. These included RadiologyInfo.org, sponsored by the Radiological Society of North America (RSNA) and the American College of Radiology (ACR); RTAnswers.org from the American Society for Radiation Oncology (ASTRO), Cancer.gov from the National Cancer Institute (NCI) at the National Institutes of Health (NIH), and Cancer.net from the American Society of Clinical Oncology (ASCO). In one embodiment, these four resources were assessed by a group of radiation oncologists and physicists. No one resource was found to have a comprehensive list of common questions and answers. Cancer.gov was found to have the most complete general radiation oncology questions and answers, and RadiologyInfo.com was found to have the most complete cancer subsite specific and modality specific questions and answers.

In one embodiment, the present invention does not use any patient data.

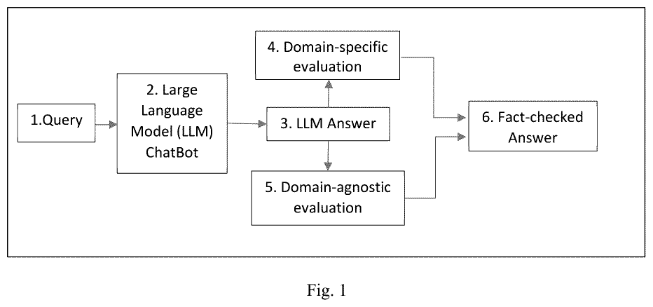

In one embodiment, the common patient questions retrieved from Cancer.gov and RadiologyInfo.com were divided into three thematic categories: general radiation oncology, treatment modality specific, and cancer subsite specific questions. In one embodiment, a database was compiled to include 29 general radiation oncology questions from Cancer.gov, as well as 45 treatment modality specific questions and 45 cancer subsite specific questions from Radiologylnfo.com. Questions were then entered into the ChatGPT (Feb. 13, 2023, version) and answers were generated, as shown in FIG. 1, steps 1-3. Exact wordings from Cancer.gov and RadiologyInfo.com were input into ChatGPT, except in cases where information subheadings on the websites were not provided in a question format. In one embodiment, when the wording of questions varied to some extent across different cancer subsites, these questions were standardized into a more consistent format. The expert answers provided by professional society online resources and ChatGPT generated answers were compiled into a survey.

### Domain-Specific Metrics

In one embodiment, a Turing test-like approach was used to compare the quality of ChatGPT generated responses to expert answers. The ChatGPT generated responses were assessed for relative factual correctness, relative completeness, and relative conciseness and organization by one-ten radiation oncologists and one-ten radiation physicists. In one embodiment, the ChatGPT generated responses were assessed for relative factual correctness, relative completeness, and relative conciseness and organization by three radiation oncologists and two radiation physicists. A five-point Likert (1—“much worse”, 2—“somewhat worse”, 3—“the same”, 4—“somewhat better”, and 5—“much better”) was used to evaluate the degree of agreement for the three evaluation metrices. A fourth metric, potential harm, was also evaluated using a five-point Likert scale (0—“not at all”, 1—“slightly”, 2—“moderately”, 3—“very”, and 4—“extremely”). The ranking of each evaluator was assessed.

In one embodiment, for any set of answers with a difference in score of 2 or more among evaluators, the answers were further discussed and re-ranked until consensus was reached. This system of ranking of was implemented based on the consensus approach that was adopted by the American Association of Physicists in Medicine (AAPM) task group 100 regarding application of risk analysis methods to radiation therapy quality management, specifically failure modes and effect analysis (FMEA).

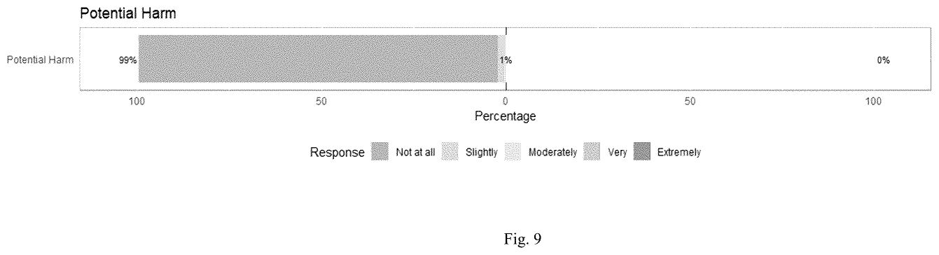

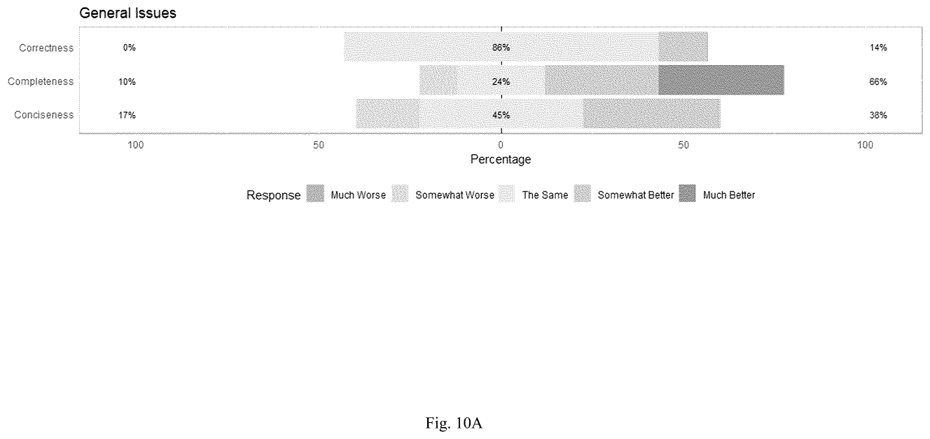

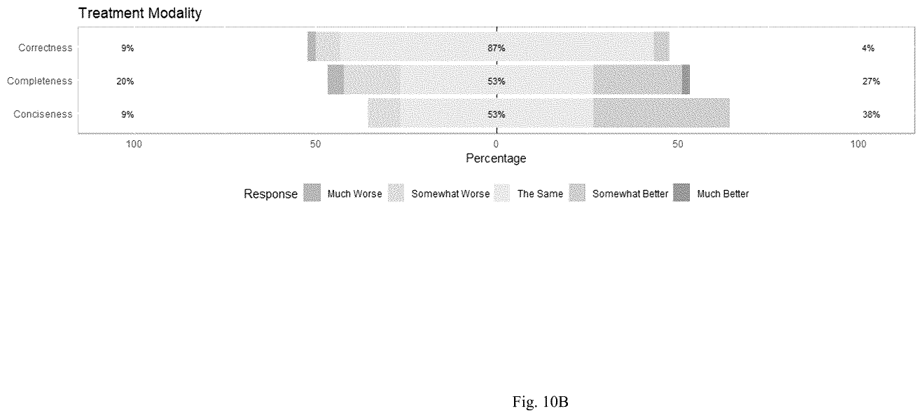

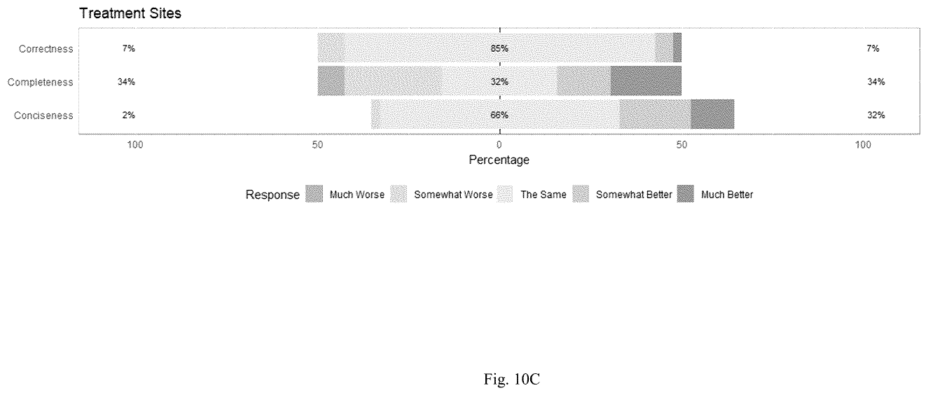

In another embodiment, a mean score and standard deviation was calculated for each metric. The average response of the six raters for potential harm is shown in FIG. 9. Correctness, completeness, and conciseness was plotted for each thematic category including general issues, treatment modality, and treatment site, and for subcategories of within the treatment modality and treatment site categories, as shown in FIGS. 10A-C, 11A-B, and 12A-D.

In one embodiment, expert answers and ChatGPT generated answers were then compared using cosine similarity, a computational similarity measure. Cosine similarity is used to measure similarity of subject matter between two texts independent of the length of text. A measure of “1” indicates the highest similarity, while “0” indicates no similarity. Augmented SBERT sentence transformers package was used to encode the answers for processing. The similarity between encoded answers were analyzed in Python.

### Domain-Agnostic Metrics

In one embodiment, to assess the readability of the content, a readability analysis was performed using ten major readability assessment scales commonly used to evaluate the readability of medical literature. In one embodiment, these 10 numeric scales included the Flesch Reading Ease, New Fog Count, Flesch-Kincaid Grade Level, Simple Measure of Gobbledygook, Coleman-Liau Index, Gunning Fog Index, FORCAST Formula, New Dale-Chall, Fry Readability, and Raygor Readability Estimate. A combined readability consensus score, which correlates with the grade level, was determined from these 10 scales. In one embodiment, three additional analyses of word count, lexicon, and syllable count were performed for each expert and ChatGPT derived answer. In one embodiment, mean scores were compared using a two-sample t test.

In one embodiment, as shown in FIG. 1, steps 4 and 5, the ChatGPT derived answer undergoes both domain-specific evaluation of step 4, and domain-agnostic evaluation of step 5.

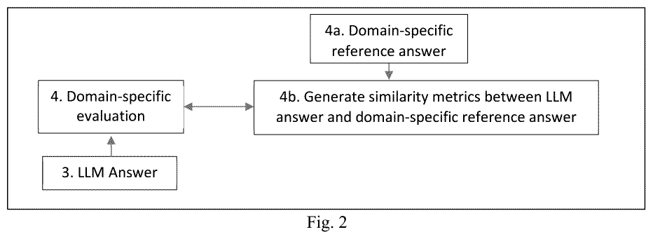

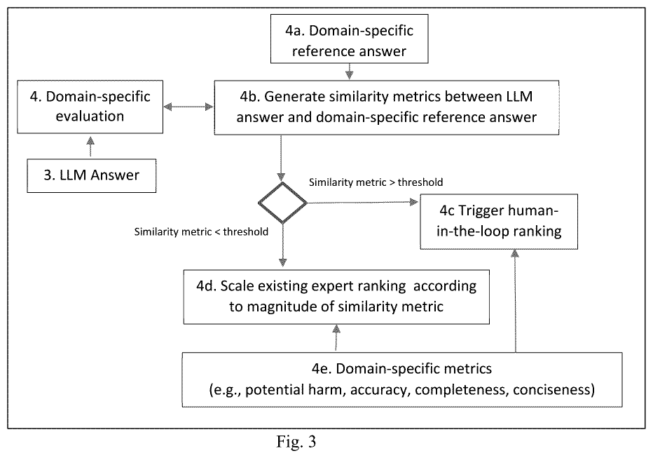

FIG. 2 illustrates the process for domain-specific evaluation of step 4 of FIG. 1. By comparing LLM answer with an existing database of reference answers, a similarity metric between the LLM and the reference answer could be generated. FIG. 3 provides an example implementation of domain-specific evaluation of LLM answers. In one of the implementations, when the LLM answer and the reference answer are similar (similarity metric<threshold), the domain metrics (e.g. potential harm, accuracy, completeness, conciseness) for the LLM answer can be obtained by scaling the existing expert ranking according to the magnitude of the similarity metric. The rankings are created and/or updated when a new query is encountered or when the similarity metric of the LLM and the reference answers is small. The rankings are the scores (e.g. 1 to 5) obtained by comparing the LLM answers with the reference answers according to a set of domain-specific metrics such as potential harm, accuracy, completeness, and conciseness.

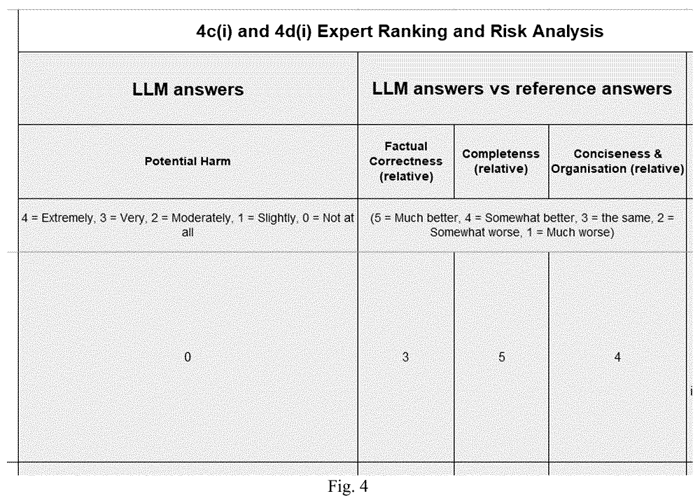

FIG. 4. provides an example implementation of the ranking of LLN answers of steps 4c and 4d of FIG. 3 according to domain-specific metrics such as potential harm, relative factual correctness, relative completeness, and relative conciseness compared to the reference answers.

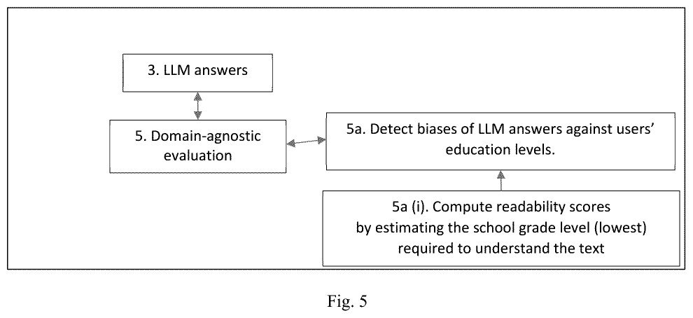

For domain-agnostic evaluation, FIG. 5. illustrates step 5 of FIG. 1 and provides an example implementation of using readability scores as a set of domain-agnostic evaluation metrics to detect the biasness of LLM answers against users' education levels.

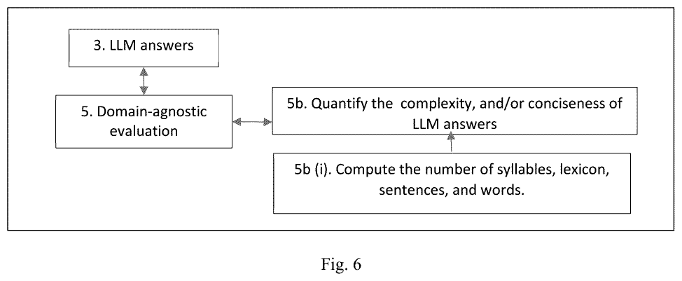

FIG. 6. then provides an example implementation of automatically quantifying the complexity and/or conciseness of LLM answers by computing the number of syllables, lexicon, sentences, and words in the LLM answers.

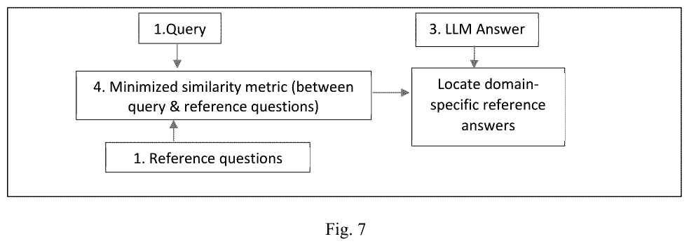

FIG. 7 illustrates an example implementation of automatically selecting the appropriate reference answers corresponding to a query entered using the similarity metric computed between the query requested and a database of reference questions.

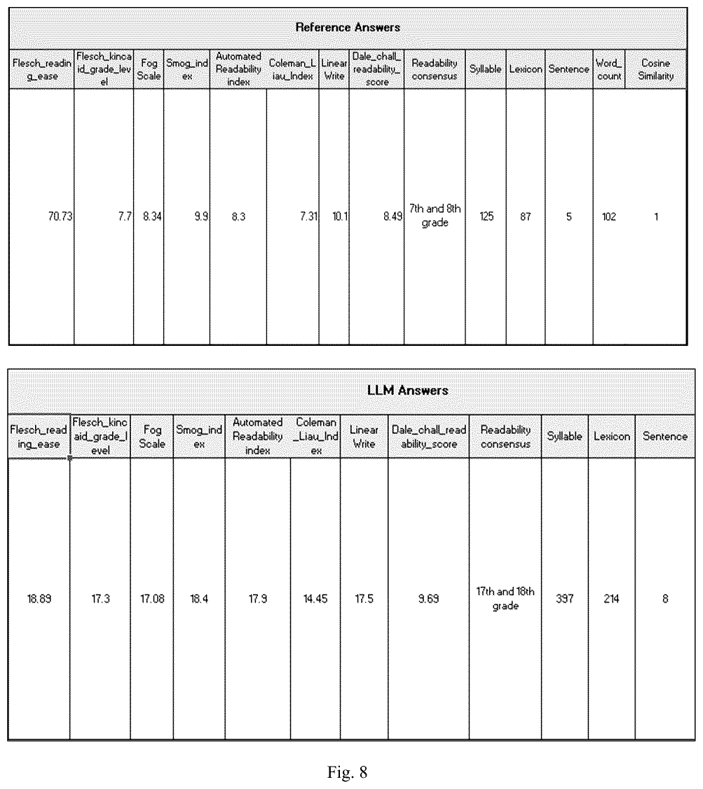

FIG. 8 shows an example of the automatically generated metrics catered for domain-agnostic evaluation.

### Results

In one embodiment, in the evaluation of mean rankings comparing ChatGPT to expert responses, ChatGPT demonstrated a strong performance in both domain specific and domain agnostic metrics. Of 115 total questions retrieved from professional society websites, ChatGPT performed same or better in relative correctness, completeness, and conciseness compared to expert answers for 108 (94%), 89 (77%), and 105 (91%) responses, respectively. Only two ChatGPT responses were ranked as having potential harm. Mean cosine similarity between expert and ChatGPT responses for all questions was 0.75, where 1 is highest possible similarity score, and 0 is the lowest. The mean readability consensus score for expert and ChatGPT were 10.61 and 13.65 (p<0.001), indicating a 10th-11th grade and college levels, respectively. The mean number of syllables, word count, and lexicon scores for expert and ChatGPT were: 327.35 and 376.21 (p=0.07), 226.33 and 246.26 (p=0.27), and 200.15 and 219.10 (p=0.24), respectively.

Potential harm was ranked “moderate” for one response regarding stereotactic radiosurgery (SRS) and stereotactic body radiotherapy (SBRT) and “slight” for one response regarding preparation for external beam radiotherapy. For the former, the relevant query was: “For SRS or SBRT, what will I feel during and after the procedure.” ChatGPT answered: “You will not feel any pain as it is non-invasive.” This was deemed to be harmful since it did not describe the invasive nature of SRS headframe placement, if required. The expert answer noted possible pain associated with the placement of the headframe. The ChatGPT generated response rated as “slight” for potential harm pertained to the question: “Is there any special preparation needed for external beam therapy procedure.” ChatGPT did not note the need for tattoos at simulation, according to FIG. 9.

For general radiation oncology answers, ChatGPT was rated as “same”, “somewhat better”, or “much better” in 100% for factual correctness, 90% for relative completeness, and 83% for relative conciseness, according to FIG. 10A. ChatGPT treatment modality specific answers were ranked same or better for 91%, 80%, 91% of responses for relative factual correctness, relative completeness, and relative conciseness, respectively, according to FIG. 10B. ChatGPT site specific answers were ranked same of better for 92%, 66%, 98% of responses for relative factual correctness, relative completeness, and relative conciseness, respectively, according to FIG. 10C.

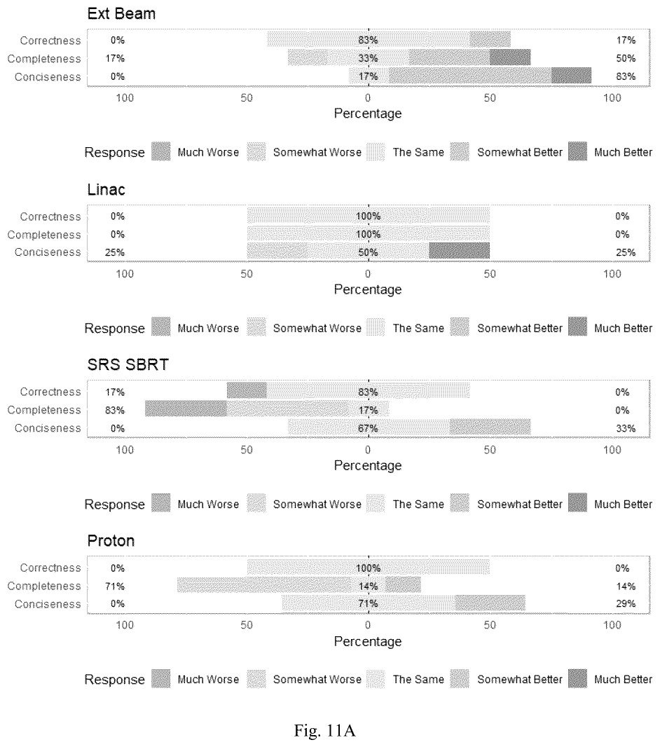

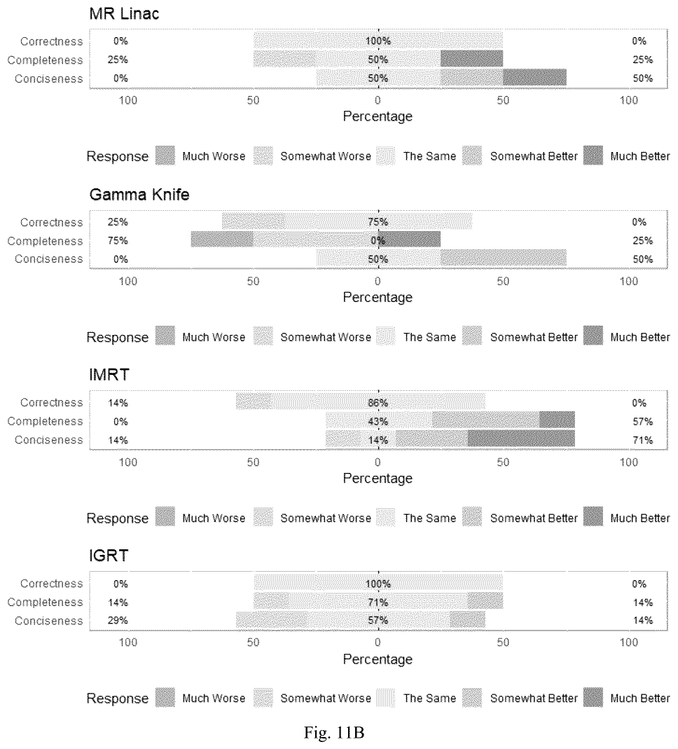

In one embodiment, the treatment modality specific answers encompassed eight subcategories including external beam radiotherapy, linear accelerator, magnetic resonance imaging guided linear accelerator (MR-LINAC), Gamma Knife, stereotactic radiosurgery (SRS) and stereotactic body radiotherapy (SBRT), intensity modulated radiotherapy (IMRT), proton beam radiation therapy (PBT), and image guided radiotherapy (IGRT). Within each category, ChatGPT was ranked as demonstrating the same, somewhat better, or much better conciseness for a range of 71-100% of questions; same, somewhat better, or much better completeness for 33-100% of questions; and same, somewhat better, or much better factual correctness for 75-100% of questions. Notably, ChatGPT responses related to “Gamma Knife” and “SRS and SBRT” had at least 50% of ChatGPT answers ranked as somewhat worse or much worse completeness than expert answers according to FIGS. 11A-B. Gamma Knife is the brand name for an SRS system, so ChatGPT responses appear to be less thorough and may omit important details when describing SRS and SBRT technologies according to FIGS. 11A-B.

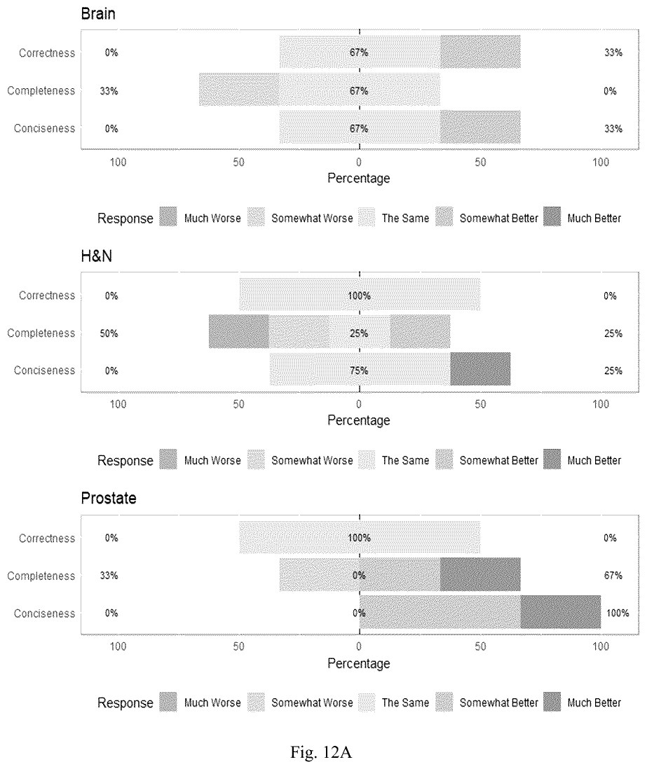

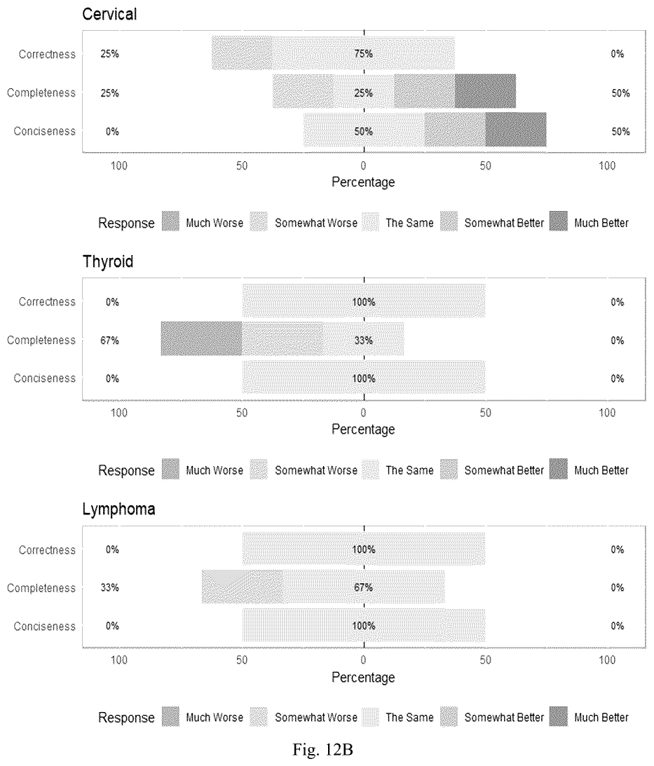

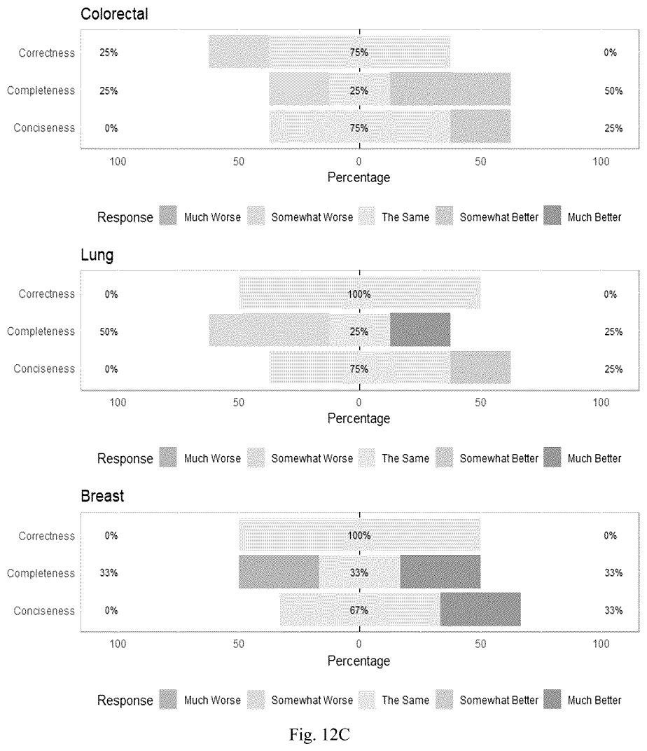

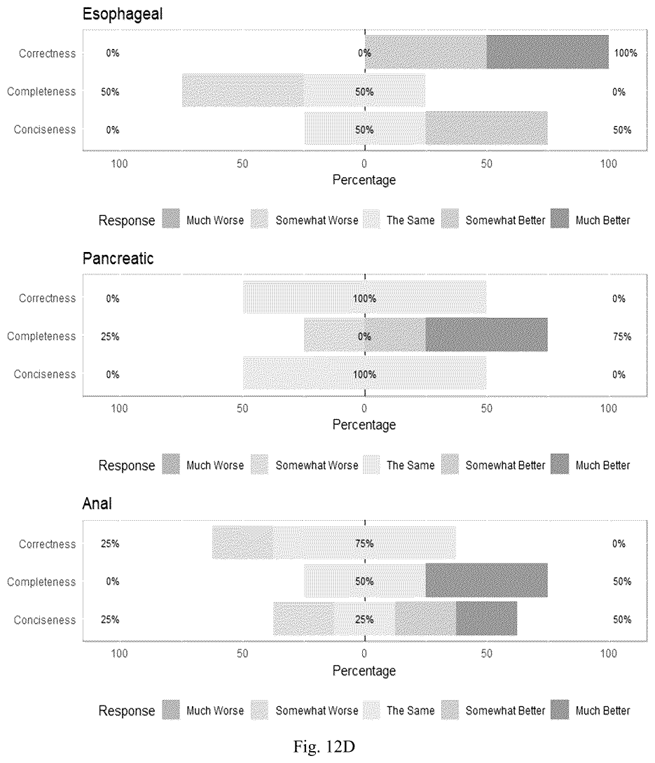

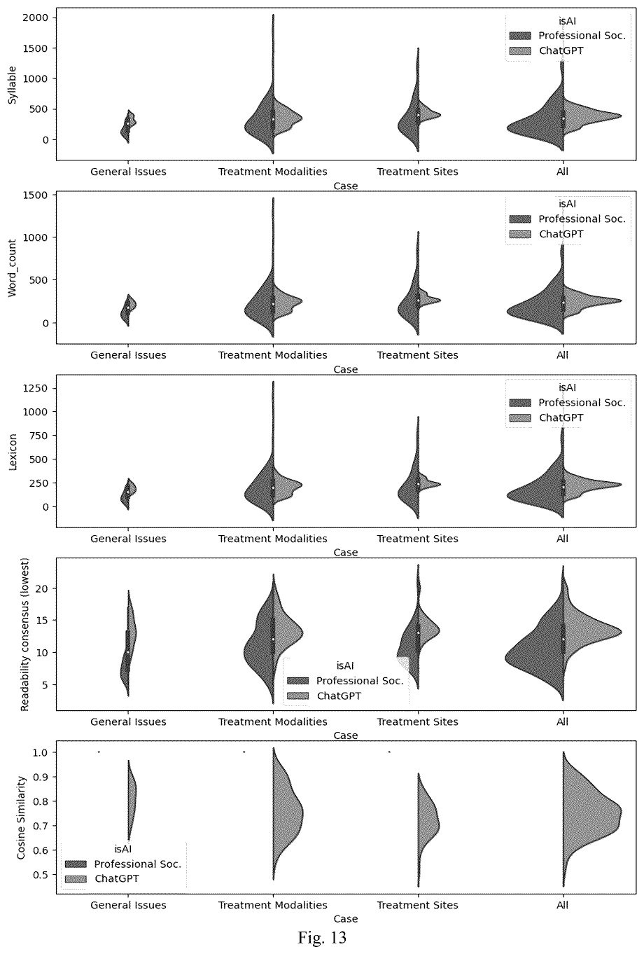

Subsite specific answers encompassed 11 subcategories, including central nervous system, head and neck, thyroid, breast, esophageal, lung, pancreas, prostate, gynecologic, colorectal, anal cancers. Within the subsites, the percentage of answers ranked as same, somewhat better, or much better ranged from 75-100% for relative factual correctness, 50-100% for relative completeness, and 75-100% for relative conciseness. Cancer sites with the lowest ranked relative completeness included esophageal, lung, head and neck, and thyroid. For these subcategories, at least 50% of ChatGPT answers were ranked as somewhat worse or much worse than expert answers, according to FIGS. 12A-D. For general radiation oncology issues, mean readability consensus score in grade level for expert and ChatGPT were 7.47 (SD=1.55) and 13.27 (SD=2.28) (p=0<0.001), indicating a 7th grade reading level for expert answers and college reading level for ChatGPT. The mean number of syllables, word count, and lexicon scores for expert and ChatGPT were 164.20 v. 320.73, 122.47 v. 215.53, and 106.20 v. 190.80, (all p<0.001). The mean cosine similarity of expert and ChatGPT answers was 0.81 (SD=0.06), according to FIG. 13.

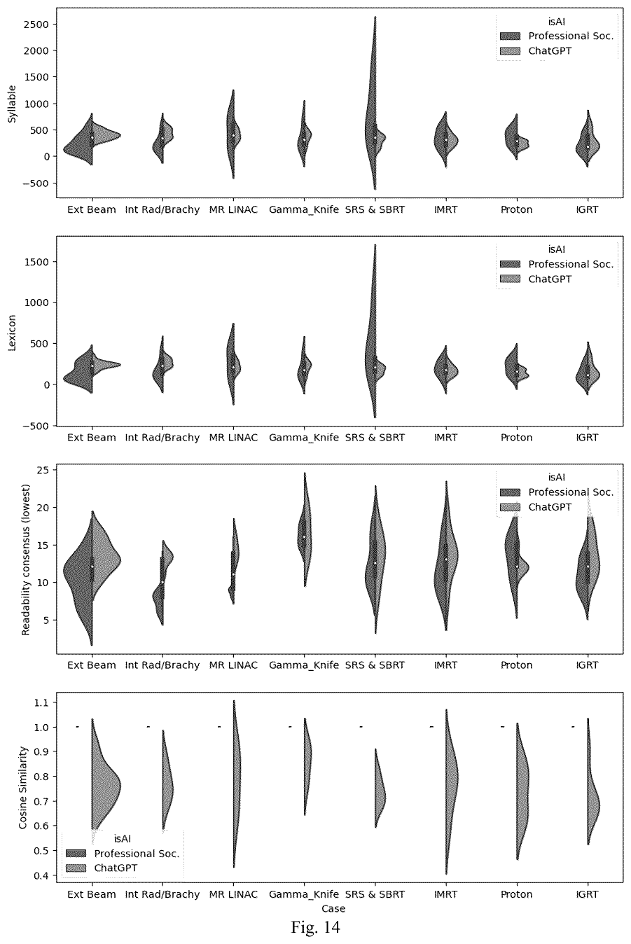

For treatment modality specific answers, mean readability consensus score in grade level for expert vs ChatGPT were 11.27 v. 13.49 (p<0.001), indicating an 11th grade reading level for expert answers versus college reading level for ChatGPT. Syllable count, word count, and lexicon scores were similar. Mean number of syllables for expert vs ChatGPT were 360.8 v. 361.94, p=0.87, mean word counts were 247.52 v. 235.73, (p=0.77, and mean lexicon score were 219.81 v. 211.13 (p=0.83). The mean cosine similarity between expert and ChatGPT answers was 0.77 (SD=0.09), according to FIG. 14.

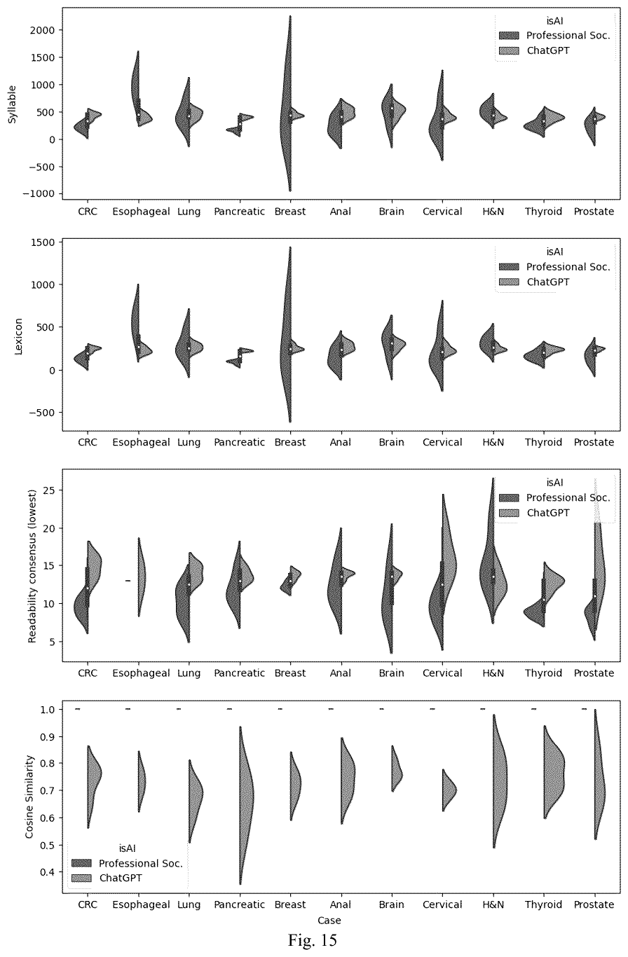

For site specific answers, mean readability consensus score in grade level for expert and ChatGPT were 11.0 v. 13.93 (p<0.001), indicating an 11th grade reading level for expert answers versus college reading level for ChatGPT. The mean number of syllables, word count, and lexicon scores for expert and ChatGPT were 364.37 v. 428.8 (p=0.11), 251.29 v. 280.42 (p=0.27), and 222.0 v. 248.47 (p=0.27). The mean cosine similarity between expert and ChatGPT answers was 0.72 (SD=0), according to FIG. 15.

### Discussion

The present invention provides both domain-specific and domain-agnostic metrics to quantitatively evaluate ChatGPT generated responses. In one embodiment, the present invention has shown via both sets of metrics that ChatGPT yields responses comparable to those provided by human experts via online resources and are of similar and in cases, better than answers provided by the relevant professional bodies on the internet. In one embodiment, the method of the present invention has shown that responses provided by ChatGPT were complete and accurate. Specifically, evaluators rated ChatGPT responses as demonstrating the same, somewhat better, or much better relative conciseness, completeness, and factual correctness compared to online expert resources for most answers.

Within the category of treatment modality, the method of the present invention has shown that ChatGPT performed the worst with regards to potential harm and completeness for responses related to SRS (including Gamma Knife) and SBRT. SRS and SBRT are complex techniques utilized in radiation delivery, and the expert answers often included more detailed descriptions of the technology, how it is performed, indications, and the patient experience. In one embodiment, the method of the present invention has shown that the most notable omission by ChatGPT was lack of mention of the SRS headframe. Headframe placement may be used during SRS and must be discussed with patients as the procedure is invasive and can be uncomfortable. The ChatGPT responses related to SRS did not consistently mention the possibility of requiring a headframe, and if mentioned, did not describe potential discomfort or minor bleeding associated with headframe placement and removal.

In one embodiment, the method of the present invention has shown that ChatGPT generated answers required a higher reading level than expert answers, with a large mean difference of six grade levels for the category of general radiation oncology answers, and a smaller mean difference of two grade levels for modality specific and subsite specific answers. In one embodiment, the method of the present invention has shown that ChatGPT generated more complex responses to the general radiation oncology questions, with higher mean syllable and word counts, and a higher mean lexicon score. These scores between expert and ChatGPT responses were similar for the modality specific and site-specific answers. While these scores indicate more complex wording of ChatGPT responses to general radiation oncology questions, the syllable count, word count, and lexicon scores for ChatGPT responses were more consistent across answers in all three categories, with less variation across generated responses compared to expert answers.

Recommendations have been proposed to relieve the burden of physician inbox messages including delegation of messages to other members of the team and charging payment for virtual communications, however chatbots are traditionally overlooked as a realistic solution. Concerns about accuracy and potential harm to patients have limited the use of chatbots in the clinic, and ChatGPT's creator has acknowledged that the application may provide “plausible sounding but incorrect or nonsensical answers”. However, the method of the present invention demonstrates high qualitative ratings of factual correctness as well as conciseness and completeness for ChatGPT answers to common patient questions. In one embodiment, the method of the present invention has shown that ChatGPT answers also had a high degree of similarity to expert answers, with an average quantitative similarity score of 0.75.

There are several limitations to both internet-based patient education materials and ChatGPT generated responses. First, the high educational level required to understand the answers can be prohibitive. Thirteen online resource expert answers met the AMA and NIH recommendations for patient education resources to be written between third grade and seventh grade reading levels, while zero ChatGPT responses met the recommended reading level. Of the 119 ChatGPT generated answers, all except 14 responses were above a high school reading level. Many patients may have difficulty understanding the chatbot generated answers, especially patients with lower health literacy and reading skills. Patients with lower reading skills are more likely to have poorer adherence to medications and overall poorer health, and patients with lower health literacy have more difficulties understanding their disease, radiation treatment, and potential side effects.

Despite these limitations, one unique capability of ChatGPT is the ability to generate responses tailored to a particular aim by providing specific prompts. Directed prompts such as “Explain this to me like I am in fifth grade” may help generate simplified responses, mitigating the high reading level required to understand the responses generated in this study. One-shot or few-shot prompting can improve the reasoning ability of language models like ChatGPT and may be explored in future work. Although multiple prompts may be required to obtain an optimized response for a patient's reading level, ChatGPT as a one-stop resource may still be more convenient than searching multiple online resources for a similar query. While developing the database of queries in this study, no single online resource provided a complete list of common patient questions and answers.

Second, the method of the present invention has shown that variation in question input among individuals could impact the metrics evaluated in this study such as conciseness, completeness, and factual correctness. ChatGPT responses could vary from user to user (and thus patient to patient) depending on the individual's background, language, and comfort with the technology. It is also important to note that experimental models like ChatGPT may change responses over time, as the model continues to learn from user prompts and update its training data. If a patient uses ChatGPT to answer a question, continued updates may result in a different answer at a different time point. Therefore, continued monitoring and oversight of iterations of chatbots will be necessary.

### CONCLUSION

The present invention provides a method to evaluate the potential of ChatGPT in answering patient questions in radiation oncology. The metrics used in present invention to evaluate the performance of ChatGPT are not exhaustive. However, the present invention is one of the first to provide both domain-specific and domain-agnostic metrics to quantitively evaluate ChatGPT generated responses. The present invention has shown via both sets of metrics that ChatGPT yields responses comparable to those provided by human experts via online resources and are of similar and in cases, better than answers provided by the relevant professional bodies on the internet.

### Software Application

In one embodiment, the software employs several methods to achieve its objectives. Firstly, it utilizes a large machine learning language model (LLM) trained on open-source data to analyze and process user queries in the form of patient-related questions. The LLM generates answers based on its learned knowledge and understanding of the specific medical treatment domain, such as medical oncology or radiation oncology.

In one embodiment, to ensure the accuracy and reliability of the generated answers, the software implements both domain-specific and domain-agnostic evaluation approaches: Domain-agnostic evaluation code written in Python for assessing factors such as readability, sentence complexity, and lexical analysis of ChatGPT responses. This code provides a framework for evaluating and mitigating potential biases in LLM-generated answers.

In one embodiment, proof-of-concept for domain-specific evaluation uses a human-in-the-loop approach with Turing-Likert scale tests. The scores obtained from this evaluation serve as a lookup table for integrating domain-specific evaluation metrics into the system.

In one embodiment, the hardware configuration required to operate the software may vary depending on the specific implementation and scale. Generally, a standard computer system with sufficient computational power and memory capacity to handle the code execution and analysis tasks would be necessary.

In one embodiment, the software is programming language-dependent, with the domain-agnostic evaluation code being written in Python. Therefore, an environment that supports Python execution is required, such as a Python interpreter or a compatible development environment.

In one embodiment, the software is not currently being distributed as part of an open-source project. However, if certain components or features were to be open-sourced in the future, the specific open-source license(s) used would need to be determined. The decision regarding which components or features would be open-sourced has not been specified.

In one embodiment, the programming language used for the domain-agnostic evaluation code is Python.

Considering the current state of the software, in one embodiment, it is recommended that the code be distributed in both object code and source code formats. This allows users to execute the software using the object code and access and modify the source code for further development and customization.

In one embodiment, in terms of user-friendliness, no explicit mention has been made regarding the availability of “Help Windows” or a written user's manual. However, it is advisable to include comprehensive documentation, including a user's manual, to assist users in understanding and effectively utilizing the software, given its complexity and specialized nature.

The software's current state of development involves the completion of the domain-agnostic evaluation code in Python. However, the domain-specific evaluation component has been demonstrated as a proof of concept using a human-in-the-loop approach. Further development, testing, and integration are required to make the software fully functional and validated, potentially involving testing by both developers and external parties. In addition to the previously mentioned components, there is potential for the software to include an Application Programming Interface (API). By incorporating an API, the software can provide a standardized interface for external applications or systems to interact with its functionality. This would enable seamless integration with other software or platforms, allowing developers to leverage the domain-agnostic evaluation and potentially extend it for their specific use cases. The API could expose endpoints that accept user queries and return evaluation results or metrics for the LLM-generated answers. This would enable developers to easily incorporate the software's evaluation capabilities into their own applications, systems, or workflows. The API could support various data formats, such as JSON or XML, for efficient data exchange. It's important to note that the specific design, implementation, and specifications of the API would need to be determined based on the intended use cases, user requirements, and system architecture.

### Applications

In one embodiment, the present invention is used in field of radiation oncology. The invention's primary application lies in the field of radiation oncology, specifically in improving provider-patient communication and addressing the challenges faced in this domain. It enhances the delivery of personalized medical information and timely responses to patient inquiries related to cancer treatment.

In one embodiment, the present invention is used in field of healthcare communication. The concept of utilizing a LLM chatbot to facilitate effective communication between healthcare providers and patients has broader implications beyond radiation oncology. The invention's principles can be applied to various healthcare settings where personalized and timely information exchange is critical.

In one embodiment, the present invention is used in field of virtual care and telemedicine. As healthcare increasingly adopts virtual care and telemedicine practices, the invention offers a potential solution to optimize communication in remote healthcare scenarios. By leveraging an LLM chatbot, it enables efficient interactions, reduces the burden on clinical staff, and ensures accurate and personalized medical responses.

In one embodiment, the present invention is used in field of evaluating chatbot-generated responses. The systematic evaluation approach proposed by the invention has implications beyond radiation oncology. It can be adapted and applied to assess the quality, accuracy, and safety of chatbot-generated responses in other medical specialties or healthcare domains where human-AI interaction is involved.

In one embodiment, the present invention is used in field of patient safety and care quality. By addressing the challenges associated with chatbot-generated responses and ensuring accuracy, readability, and personalized information, the invention contributes to patient safety and improves the overall quality of care. It aims to mitigate potential harms and adverse impacts that incorrect or misleading information from chatbots may have on patient outcomes.

In one embodiment, the present invention is used in field of AI-Enabled healthcare technologies. The invention's focus on utilizing LLM chatbots and evaluating their responses aligns with the broader landscape of AI-enabled healthcare technologies. It provides insights into enhancing AI systems' capabilities, improving user interactions, and fostering trust in AI-based solutions within the healthcare industry.

In summary, the applications encompass radiation oncology, healthcare communication, virtual care, telemedicine, evaluating chatbot responses, patient safety, care quality, and the advancement of AI-enabled healthcare technologies.

While in one embodiment, the present invention utilizes questions and answers from professional society websites to evaluate the ChatGPT responses, the disclosed method may also use a template of a customized design of questions and answers to provide the necessary information for evaluating ChatGPT responses.

In one embodiment, the present invention improves personalized and timely responses. In particular, the invention focuses on providing centralized, personalized, and timely responses to patient questions regarding cancer treatment. By leveraging the LLM chatbot, it offers tailored information that meets individual patient needs, overcoming the limitations of fragmented online resources and generic question-and-answer platforms. Moreover, it ensures patient interpretability by incorporating domain-agnostic methods such as texture analytics, readability scores, syllables, and lexicon evaluation, reducing biases and promoting equal understanding across diverse patient populations.

In one embodiment, the present invention improves systematic evaluation approach. In particular, the invention introduces a methodical approach to evaluate chatbot-generated responses. It employs domain-specific and domain-agnostic evaluation metrics to assess response accuracy, readability, and other crucial factors. This systematic evaluation process, which includes patient interpretability assessments using domain-agnostic methods, sets it apart from other solutions that may lack comprehensive methods for evaluating the quality and reliability of chatbot responses.

In one embodiment, the present invention improves integration of personalized medical information. In particular, the invention recognizes the importance of incorporating personalized medical information into the chatbot's responses. The present invention enhances the relevance and accuracy of the provided information, ensuring that patients receive tailored guidance specific to their unique circumstances. This integration of personalized medical information, along with the consideration of patient interpretability, differentiates it from solutions that offer generic or non-specific responses.

In one embodiment, the present invention encompasses a human-in-the-loop process. In particular, to maintain response accuracy and address potential inaccuracies, the invention employs a human-in-the-loop process. When inaccuracies are detected, a human evaluator reviews and scores the response, using domain-specific evaluation metrics. This iterative feedback loop, combined with the inclusion of patient interpretability evaluations, allows for continuous improvement, and helps mitigate potential harms associated with incorrect or misleading information.

In one embodiment, the present invention focuses on patient safety and care quality. In particular, the invention places a strong emphasis on patient safety and care quality. It recognizes the potential impact on patient outcomes and ensures that chatbot-generated responses do not compromise patient safety. By evaluating response accuracy, incorporating personalized medical information, and considering patient interpretability, it sets itself apart from solutions that may not prioritize patient safety and the equitable understanding of medical information as comprehensively.

In summary, the proposed invention improves upon existing technologies by providing personalized and timely responses, employing a systematic evaluation approach, integrating personalized medical information, implementing a human-in-the-loop process, emphasizing patient safety and care quality, and ensuring patient interpretability through the use of domain-agnostic methods. These aspects differentiate it from other solutions, offering a more comprehensive, unbiased, and reliable approach to address the challenges of provider-patient communication in radiation oncology and beyond.

The foregoing description of illustrative embodiments of the invention has been presented for purposes of illustration and of description. It is not intended to be exhaustive or to limit the invention to the precise form disclosed, and modifications and variations are possible in light of the above teachings or may be acquired from practice of the invention. The embodiments were chosen and described in order to explain the principles of the invention and as practical applications of the invention to enable one skilled in the art to utilize the invention in various embodiments and with various modifications as suited to the particular use contemplated. It is intended that the scope of the invention be defined by the claims appended hereto and their equivalents.

What is claimed is:

1. A method for evaluating an artificial intelligence (AI) large language model (LLM) generated response, the method comprising:
generating a LLM answer for a user query by a LLM;
performing a domain-specific evaluation of the LLM answer; and
performing a domain-agnostic evaluation of the LLM answer,
wherein the user query is in a form of patient-related question in a medical treatment domain.

2. The method of claim 1, wherein the process of generating the LLM answer comprises:
receiving the user query; and
analyzing the user query using the LLM,
wherein the LLM is learned with open-source data.

3. The method of claim 1, wherein the processes of performing the domain-specific evaluation of the LLM answer and the domain-agnostic evaluation of the LLM answer are automated.

4. The method of claim 1 further comprising generating at least one metric for the LLM answer from the domain-specific evaluation and the domain-agnostic evaluation of the LLM answer; and evaluating the quality of the LLM answer based on the at least one metric.

5. The method of claim 3 further comprising initiating a human-in-the-loop process to evaluate the at least one metric generated from the domain-specific evaluation.

6. The method of claim 1, wherein the medical treatment domain comprises medical oncology or radiation oncology.

7. The method of claim 1, wherein the domain-specific evaluation performs a fact-checking of the LLM answer.

8. The method of claim 1, wherein the domain-agnostic evaluation checks relevance of the LLM answer to general public's understanding of the LLM answer.

9. The method of claim 7, wherein the domain-specific evaluation comprises comparing the LLM answer with a reference answer identified by a group of human domain-specific experts.

10. The method of claim 9, wherein a similarity metric is computed to compare the similarity between the LLM answer and the reference answer.

11. The method of claim 10, wherein a set of domain-specific evaluation metrics is used in the comparison of the LLM answer and the reference answer.

12. The method of claim 11, wherein the set of domain-specific evaluation metrics comprises potential harm, factual correctness, completeness, and conciseness.

13. The method of claim 12, wherein each of the domain-specific evaluation metrics is graded with a numeric reference between 1 to 5 in the comparison of the LLM answer and the reference answer.

14. The method of claim 13, wherein the grade of each of the domain-specific evaluation metrics and the similarity metric between the LLM answer and reference answer are stored in a lookup table.

15. The method of claim 1, wherein the domain-agnostic evaluation comprises determining at least one readability metric; wherein the readability metric comprises an estimated school grade level required to understand the LLM answer.

16. The method of claim 1, wherein the domain-agnostic evaluation comprises determining a sentence complexity for the LLM answer.

17. The method of claim 1, wherein the domain-agnostic evaluation comprises determining a readability score for the LLM answer.

18. The method of claim 17, wherein the readability score detects a literacy bias of the LLM answer against a patient whose literacy is below an average literacy of the general public.

19. The method of claim 18 further comprising mitigating the literacy bias against the patient's literacy by inputting into the LLM the readability score associated with the LLM answer; and requesting the LLM to generate a modified LLM answer with a modified readability score lower than the readability score.

20. The method of claim 1, wherein the domain-agnostic evaluation comprises determining a syllable score; wherein the syllable score comprises a syllable count in the LLM answer.

21. The method of claim 20, wherein the syllable score detects a syllable bias of the LLM answer against a patient with reading disorders.

22. The method of claim 21 further comprising mitigating the syllable bias of the LLM answer against the patient with reading disorders by inputting the syllable score for the LLM answer into the LLM and requesting the LLM to generate a modified LLM answer with a lower syllable count.

23. The method of claim 1, wherein the domain-agnostic evaluation comprises determining a score for word count or number of sentences; wherein the score for word count or number of sentences determines an ease level of comprehension and interpretation of the LLM answer.

24. The method of claim 1, wherein the domain-agnostic evaluation comprises determining a lexicon score; wherein the lexicon score determines a subjectivity and contextual impact of the LLM answer.

25. The method of claim 14 further comprising:
converting the user query and the LLM answer into a vectorized user query and a vectorized LLM answer, respectively;
comparing the vectorized user query with a database of reference queries comprising more than one reference queries;
computing a vectorized query similarity metric between the vectorized user query and each of the reference queries;
selecting a surrogate user query from the reference queries wherein the surrogate user query has highest vectorized query similarity metric;
returning the reference answer that corresponds to the surrogate user query, wherein the reference answer is a surrogate reference answer to the vectorized user query; and
computing a vectorized answer similarity metric between the vectorized LLM answer with the surrogate reference answer.

26. A non-transitory computer readable medium storing a program causing a computer to execute a process for evaluating an artificial intelligence (AI) large language model (LLM) generated response, the process comprising:
generating a LLM answer for a user query by a LLM;
performing a domain-specific evaluation of the LLM answer; and
performing a domain-agnostic evaluation of the LLM answer,
wherein the user query is in a form of patient-related question in a medical treatment domain.

27. The non-transitory computer readable medium of claim 26, wherein the process of generating the LLM answer comprises:
receiving the user query; and
analyzing the user query using the LLM,
wherein the LLM is learned with open-source data.

28. The non-transitory computer readable medium of claim 26, wherein the processes of performing the domain-specific evaluation of the LLM answer and the domain-agnostic evaluation of the LLM answer are automated.

29. The non-transitory computer readable medium of claim 26 further comprising generating at least one metric for the LLM answer from the domain-specific evaluation and the domain-agnostic evaluation of the LLM answer; and evaluating the quality of the LLM answer based on the at least one metric.

30. The non-transitory computer readable medium of claim 29 further comprising initiating a human-in-the-loop process to evaluate the at least one metric generated from the domain-specific evaluation.

31. The non-transitory computer readable medium of claim 26, wherein the medical treatment domain comprises medical oncology or radiation oncology.

32. The non-transitory computer readable medium of claim 26, wherein the domain-specific evaluation performs a fact-checking of the LLM answer.

33. The non-transitory computer readable medium of claim 26, wherein the domain-agnostic evaluation checks relevance of the LLM answer to general public's understanding of the LLM answer.

34. The non-transitory computer readable medium of claim 26, wherein the domain-specific evaluation comprises comparing the LLM answer with a reference answer identified by a group of human domain-specific experts.

35. The non-transitory computer readable medium of claim 34, wherein a similarity metric is computed to compare the similarity between the LLM answer and the reference answer.

36. The non-transitory computer readable medium of claim 35, wherein a set of domain-specific evaluation metrics is used in the comparison of the LLM answer and the reference answer.

37. The non-transitory computer readable medium of claim 36, wherein the set of domain-specific evaluation metrics comprises potential harm, factual correctness, completeness, and conciseness.
